(19) United States
(12) Patent Application Publication
(10) Pub. No.: US 2017/0209900 A1
(43) Pub. Date: Jul. 27, 2017

(54) ULTRASONIC INDUCED ARTIFICIAL BLACK HOLES IN PHONONIC CRYSTALS

(71) Applicant: FloDesign Sonics, Inc., Wilbraham, MA (US)

(72) Inventors: Bart Lipkens, Hampden, MA (US); Edward Rietman, Nashua, NH (US)

(21) Appl. No.: 15/418,473

(22) Filed: Jan. 27, 2017

Related U.S. Application Data
(60) Provisional application No. 62/287,539, filed on Jan. 27, 2016.

(51) Int. Cl. B06B 1/06; C02F 1/40; C02F 1/36; H01L 41/187; C07K 1/14; B01J 19/10; H01L 41/09; B01D 17/02; B01D 43/00

(52) U.S. Cl. CPC B06B 1/0648; B01D 17/02; C02F 1/40; C02F 1/36; B01D 43/00; C07K 1/14; B01J 19/10; H01L 41/09; H01L 41/1876; C02F 2101/32

(57) ABSTRACT

Acoustophoretic devices and methods for using such devices in various applications are disclosed. The devices include a flow chamber having an inlet; a phononic crystal within an active volume of the flow chamber; and ultrasonic transducer(s) that create an acoustic standing wave in the active volume. This combination results in the creation of high-pressure nodes within the active volume, having a value of at least 50 MPa, which is useful for different applications.

### CROSS-REFERENCE TO RELATED APPLICATIONS

This application claims priority to U.S. Provisional Patent Application Ser. No. 62/287,539, filed on Jan. 27, 2016, the disclosure of which is hereby fully incorporated by reference in its entirety.

### BACKGROUND

An ultrasonic black hole, as described herein, is essentially a very high-pressure node that, with respect to light, acts as a black hole and warps optical space. These high-pressure nodes have a number of applications, as outlined herein.

### BRIEF DESCRIPTION

The present disclosure relates, in various embodiments, to acoustophoretic systems and devices that exploit phenomena observed in phononic crystals (e.g., by inducing high-pressure modulations in a phononic crystal due to constructive and deconstructive interference using acoustic standing waves). More particularly, the devices include a flow chamber containing at least one ultrasonic transducer that is arranged to surround the phononic crystal. High-pressure nodes are then formed within the flow chamber. The systems can be used for separation of a secondary phase/particulates (e.g., bacteria, metals, salts, organics, biomolecules, proteins, algae, viruses, microcarriers, or biological cells, such as Chinese hamster ovary (CHO) cells, NSO hybridoma cells, baby hamster kidney (BHK) cells, or human cells, T cells, B cells, NK cells) from a host fluid; or for performing sonochemistry; or for denaturation of suspended proteins; or as a cosmological model.

In particular embodiments, an acoustophoretic device comprises a flow chamber; at least one ultrasonic transducer; and a reflector. The flow chamber has at least one inlet, and may have at least one outlet. At least one ultrasonic transducer surrounds at least a portion of an active volume within the flow chamber. An ultrasonic transducer may include a piezoelectric material, which may be in the form of a piezoelectric poly-crystal or ceramic poly-crystal, which may be referred to herein collectively as a crystal. The transducer can be driven to create an acoustic standing wave in the active volume within the flow chamber. The driving signal for the transducer may be based on voltage, current, magnetism, electromagnetism, capacitive or any other type of signal to which the transducer is responsive. A phononic crystal is located within the active volume, but does not occupy the entire active volume. The phononic crystal is a periodic structure that interacts with a fluid that is present within the flow chamber and the active volume (e.g. by flowing the fluid through the flow chamber), affecting the acoustic waves passing through the fluid (such as a fluid/cell mixture) that are generated by the ultrasonic transducer(s).

The phononic crystal may be in the form of a frame that supports a periodic array of objects formed from a material having a specific acoustic impedance that is very different from the fluid, e.g. greater than 15×10⁵ g/cm²·sec, which is very different from water. The periodic array may be in the form of a linear hexagonal array or a cubic array.

Generally, the phononic crystal and the fluid have different specific acoustic impedances. Thus, in some embodiments, the objects, which may be specifically shaped objects such as spheres or polygons, may be made of steel or another metal, or ceramic. The frame is made of a material having a specific acoustic impedance similar to the fluid, e.g. less than 4×10⁵ g/cm²·sec. Put another way, the frame should have a similar acoustic impedance to the fluid.

The at least one ultrasonic transducer may be a single tubular ultrasonic transducer. Alternatively, or in addition, a plurality of ultrasonic transducers can be used to surround the active volume. Acoustic standing waves are uniformly generated throughout the active volume along its longitudinal axis. A combination of ultrasonic transducers and reflectors can also be used to surround the active volume.

In some embodiments of the acoustophoretic device, the flow chamber further comprises at least one outlet. In more particular embodiments, the at least one inlet is located at a first end of the flow chamber, and the at least one outlet is located at a second end of the flow chamber opposite the first end. Such embodiments may be used to continuously flow a fluid or mixture of fluid with suspended particles/droplets through the flow chamber and the active volume for applications such as separation.

An acoustic transfer medium may be present within the active volume. In particular embodiments, the acoustic transfer medium is glycerin or water.

In other embodiments, the acoustophoretic device further comprises a first optical window located at a first end of the flow chamber, and a second optical window located at a second end of the flow chamber opposite the first end. The acoustophoretic device may further comprise a laser located so as to illuminate the active volume through the first optical window; and/or a collimator located so as to receive light through the second optical window. The collimator may be coupled to a spectrometer. The acoustophoretic device can also further comprise a beam expander between the laser and the first optical window. Such embodiments may be useful for applications where the device is used as an analogy for various cosmological models.

Methods for separating a secondary phase or a particulate from a primary phase or host fluid are disclosed. The methods comprise flowing a mixture of the host fluid and the secondary phase or particulate through an acoustophoretic device according to the present disclosure, such as the acoustophoretic devices previously described. This fluid mixture fills the remainder of the active volume not occupied by the phononic crystal. The methods further comprise driving the ultrasonic transducer to create the acoustic standing wave. The ultrasonic transducer may be driven by an electrical signal, which may be controlled based on voltage, current, phase angle, power, frequency or any other electrical signal characteristic. The secondary phase or particulate is driven to high pressure nodes in the active volume based on their acoustic contrast factor, wherein the high pressure nodes have a pressure of at least 50 MPa. Proteins can be separated from a cell-protein matrix suspended in a cell culture medium. Oil-water mixtures can also be separated. Due to the high pressures that are involved in a phononic crystal, proteins may also be denatured, that is the protein loses the quaternary structure, tertiary structure, and secondary structure which is present in their native state.

In particular embodiments, the acoustic standing wave is a multi-dimensional acoustic standing wave. The multi-dimensional acoustic standing wave can result in an acoustic radiation force having an axial force component and a lateral force component that are of the same or different order of magnitude.

Methods of performing sonochemistry may also be conducted in the acoustophoretic devices described above. A mixture of a host fluid and at least two reactants is flowed/placed into the flow chamber and the active volume. When the transducer(s) are exited or driven to create acoustic standing waves within the active volume, a chemical reaction between the reactants occurs. The transducer(s) may be driven by an electrical signal, such as a voltage signal.

High-pressure nodes may also be useful for other applications, such as denaturation of suspended proteins. Generally, an acoustic transfer fluid is placed within the flow chamber and the active volume, and the acoustic standing waves are generated within the active volume.

Ultrasonic standing waves in a cylindrical enclosure can achieve very high local pressures at the acoustic nodes (˜1 MPa). When the acoustic wave passes through a phononic crystal, such as one comprised of steel and glycerin, there are regions of constructive interference resulting in even higher local pressures (˜70 MPa). At these localized high pressure points inside the phononic crystal, conventional acoustics equations fail, and the mathematics of gravitational fields are more appropriate. These pressures are so high as to create optical space singularities, analogous to cosmological phenomena. A redshift for light passing through this system is measured and how the optical space can thus be created is described by elementary equations of a non-rotating black hole.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a graph of a two-dimensional Bessel function showing the variation of the amplitude of the acoustic pressure in both radius (in a cylindrical transducer) and time. The radius (r) is illustrated along an axis running from −10 to +10 in intervals of 5. The time (t) is illustrated along an axis running from 0 to 10 in intervals of 5. The amplitude is arbitrarily set to 5, the wave number is arbitrarily set to 1, and the angular frequency is arbitrarily set to 3.

FIG. 2 is a cross-sectional illustration of two different finite element simulations of steel balls in glycerin surrounded by a circular acoustic transducer. The circular structure on the left-hand side shows a ring transducer packed with steel balls and glycerin. The steel balls are arranged in a cubic array. The circular structure on the right-hand side shows the same ring transducer filled with glycerin, but without any steel balls packed therein. The x-axis and y-axis are values used in the finite element simulation and are an arbitrary pressure scale.

FIG. 3 illustrates two simulations showing the hyperbolic surface similarity between Minkowski space-time (top) and optical space (bottom), with the optical space computed for glycerin as the acoustic transfer medium. In the top graph, both axes are arbitrary. In the bottom graph, the y-axis is refractive index, and the x-axis is the angle in radians.

FIG. 4 is a simulation of a three-dimensional hyperbolic surface showing warping of the space-time by large masses, computed with the Weierstrass function.

FIG. 5 is a picture of an exemplary acoustophoretic device of the present disclosure. The device includes an optical detector, a collimator, a flow chamber with optical windows located at either end, a beam expander, and a laser. The device includes a tubular acoustic transducer. The inset picture at top right shows a cylindrical frame holding an array of 5.0 mm magnetic steel balls in a hexagonal array along its longitudinal axis, which is the phononic crystal.

FIG. 6 is a picture showing laser light projected through the device of FIG. 5, when no phononic crystal is present. The transducer is operated at 1.62 MHz and 10 Vpp.

FIG. 7 is a picture showing laser light projected through the device of FIG. 5, when the phononic crystal is present.

FIG. 8 is a graph showing the spectrometer data from one of the lighted “blobs” in FIG. 7. The y-axis is intensity, and the x-axis is wavelength (nm).

FIG. 9 is a cross-sectional diagram of a conventional ultrasonic transducer.

FIG. 10 is a cross-sectional diagram of an ultrasonic transducer of the present disclosure. An air gap is present within the transducer, and no backing layer or wear plate is present.

FIG. 11 is a cross-sectional diagram of an ultrasonic transducer of the present disclosure. An air gap is present within the transducer, and a backing layer and wear plate are present.

FIG. 12 is a graph showing the relationship of the acoustic radiation force, gravity/buoyancy force, and Stokes' drag force to particle size. The horizontal axis is in microns (μm) and the vertical axis is in Newtons (N).

FIG. 13 is a graph of electrical impedance amplitude versus frequency for a square transducer driven at different frequencies.

FIG. 14A illustrates the trapping line configurations for seven peak amplitudes of an ultrasonic transducer of the present disclosure. FIG. 14B is a perspective view generally illustrating a device of the present disclosure. The fluid flow direction and the trapping lines are shown. FIG. 14C is a view from the fluid inlet along the fluid flow direction (arrow 141) of FIG. 14B, showing the trapping nodes of the standing wave where particles would be captured.

FIG. 14D is a view taken through the transducers face at the trapping line configurations, along arrow 143 as shown in FIG. 14B.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, i.e. the surface of the earth. The terms “upwards” and “downwards” are also relative to an absolute reference; upwards is always against the gravity of the earth.

The term “parallel” should be construed in its lay sense of two surfaces that maintain a generally constant distance between them, and not in the strict mathematical sense that such surfaces will never intersect when extended to infinity.

The present application may refer to “the same order of magnitude.” Two numbers are of the same order of magnitude if the quotient of the larger number divided by the smaller number is a value of at least 1 and less than 10.

The present disclosure relates to acoustophoretic devices and their use in multiple applications. Generally, an acoustic standing wave generates pressure minima at locations on the standing wave where the amplitude is minimum and maximum. These are called, respectively, nodes and anti-nodes. These pressure minima nodes and anti-nodes may be utilized to capture materials that are differentiated from the surrounding environment by size, density and compressibility (i.e., the speed of sound through the material). Those materials that collect at the pressure minima nodes are known as having a positive contrast factor. Those materials that collect at the pressure minima anti-nodes are known as having a negative contrast factor. In the devices disclosed herein, especially high pressures can be generated, of at least 50 MPa, up to 70 MPa, and even up to 100 MPa.

Inside such an acoustophoretic device, a standing wave was obtained that was best described by Bessel functions of the first kind, where the pressure is a function of radius and time:

p(r,t)=AJ₀(kr)sin(ωt) (1)

where J₀ is a Bessel function; A is the acoustic pressure amplitude, arbitrarily set to 5; k is the wavenumber, arbitrarily set to 1; and ω is the angular frequency, arbitrarily set to 3.

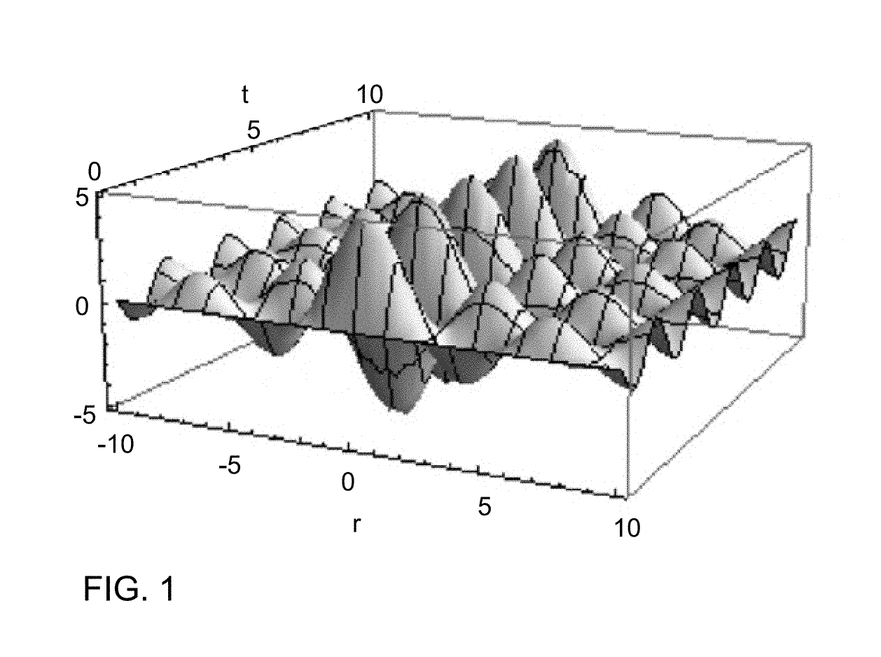

A plot of the function is presented in FIG. 1, which shows a two-dimensional Bessel function showing the amplitude of the acoustic pressure in both radius (in a cylindrical transducer) and time. Like all Bessel functions, the central peak has the strongest amplitude. Beyond the central peak, the peaks have decreasing amplitude, or are damped, with respect to the radius, and oscillations in time (i.e., the plot of the wave appears as a damped oscillator). As will be explained in greater detail herein, this behavior is observed in a tubular acoustic transducer. The acoustic pressure amplitude is a function of the applied voltage and the characteristics of the transducer.

If, as shown in FIG. 1, there are no obstructions blocking the free standing wave, the modulations of the fluid within the confines of the tube modulate the density of the fluid. These modulations of the fluid may cause refractive index modulations. These refractive index modulations, as are shown herein, can be quite extreme and warp optical space in ways analogous to black holes and galaxies warping space-time.

When an advancing wave front meets a material of very high impedance, it will tend to increase its phase velocity through that medium. Likewise, when the advancing wave front meets a low impedance medium, it will slow down. This concept can be exploited with periodic arrangements of impedance-mismatched elements to affect acoustic waves in a crystal—a phononic crystal.

For inhomogeneous solids, the elastic wave equation is given by

$$\frac{{\partial }^{2} {u}_{j}^{i}}{\partial t} = \frac{1}{ρ} \{ \frac{\partial }{\partial {x}_{i}} ( λ \frac{\partial {u}_{j}^{i}}{\partial {x}_{l}} ) + \frac{\partial }{\partial {x}_{l}} [ μ ( \frac{\partial {u}_{j}^{i}}{\partial {x}_{l}} + \frac{\partial {u}_{j}^{i}}{\partial {x}_{i}} ) ] \} \qquad ( 2 )$$

where uᵢ is the i^{th} component displacement vector; the subscript j is the reference to the medium (medium 1 or medium 2); μ and λ are known as the Lame coefficients; ρ is the density; and the longitudinal speed of sound (cₗ) and transverse speed of sound (cₜ) are given by:

cₗ=\sqrt{(λ+2μ)/ρ}

cₜ=\sqrt{μ/ρ}

and where the Lame coefficients can be expressed as Young's modulus E:

Eₜ=ρcₜ²=μ

Eₗ=ρcₗ²=λ+2μ

In a linear standing wave (not in a tubular device), the pressure nodes from the acoustic wave produce an acoustic radiation force F_{ac} on the particles/secondary phase elements (e.g., proteins) according to

$${F}_{a c} = X π {R}_{p}^{3} k \frac{{A}^{2}}{{ρ}_{f} {c}_{f}^{2}} sin ( 2 kx ) \qquad ( 3 )$$

where Rₚ is the particle radius, p is the acoustic pressure, k is the wave number, x is the position, p is the fluid density, c is the speed of sound in that fluid phase, A the maximum amplitude of the acoustic pressure as given in Eq. (1), and X is the contrast factor given by

$$X = \frac{1}{3} ( \frac{5 Λ - 2}{1 + 2 Λ} - \frac{1}{{σ}^{2} Λ} )$$

where Λ is the ratio of particle density to fluid density and σ is the ratio of particle sound speed to fluid sound speed.

The theoretical model that is used to calculate the acoustic radiation force is the formulation developed by Gor'kov, where the acoustic radiation force F_{ac} is defined as a function of a field potential U, F_{ac}=−∇ (U), where the field potential U is defined as

$$U = {V}_{O} [ \frac{〈 {p}^{2} ( x , y , t ) 〉}{2 {ρ}_{f} {c}_{f}^{2}} {f}_{1} - \frac{3 {ρ}_{f} 〈 {v}^{2} ( x , y , t ) 〉}{4} {f}_{2} ] \qquad ( 4 )$$

where f₁ and f₂ are the monopole and dipole contributions defined by

$${f}_{1} = 1 - \frac{1}{Λ {σ}^{2}} \qquad {f}_{2} = \frac{2 ( Λ - 1 )}{2 Λ + 1}$$

and where

$$σ = \frac{{c}_{p}}{{c}_{f}} \qquad Λ = \frac{{p}_{p}}{{p}_{f}} \qquad {β}_{f} = \frac{1}{{ρ}_{f} {c}_{f}^{2}}$$

where ρ is the acoustic pressure, u is the fluid particle velocity, Λ is the ratio of cell density ρₚ to fluid density ρ_{f}, σ is the ratio of cell sound speed cₚ to fluid sound speed c_{f}, Vₒ=πRₚ³ is the volume of the cell, and < > indicates time averaging over the period of the wave.

For purposes of the present disclosure, interest in this function lies where p→∞, essentially a singularity, then the acoustic field U→∞. While this state is an unrealistic situation under normal conditions, because of the constructive interference, the pressures can become very large inside the phononic crystal, though not technically infinite. In fact, the pressures and field force become so large that the conventional theory breaks down. Essentially, what occurs is a singularity best described by theories of cosmology, namely the physics of black holes.

One construction of a phononic crystal may be in the form of a meta-material. A meta-material is a material engineered to have a property that is not found in nature. These type of materials utilize multiple elements utilizing a composition of materials such as a plastic. The repeating pattern of the material, that is typically smaller than the wavelength of the phenomena that the material is influencing, generate the properties of the meta-material. It is in fact the structure of the meta-material, not the base material, that gives the meta-material its specialized properties. Examples of meta-materials include honeycombed formed plastics and tessellation patterns of 3-D materials.

To describe the metric signature transitions in meta-materials, the Klein-Gordon eigen-equation of motion in a quantum scalar field on some space-time is:

$$\frac{{\partial }^{2} ϕ}{\partial {x}_{i}^{2}} = {m}^{2} ϕ$$

This is a momentum equation and can be written for a flat (i.e., 2+2) four-dimensional 2T space-time as the expanded Klein-Gordon equation in coordinate space as:

$$( \frac{{\partial }^{2}}{\partial {x}_{1}^{2}} + \frac{{\partial }^{2}}{\partial {x}_{2}^{2}} + \frac{{\partial }^{2}}{\partial {x}_{3}^{2}} + \frac{{\partial }^{2}}{\partial {x}_{4}^{2}} ) ϕ = 0 \qquad ( 5 )$$

and in momentum or k-space as (−k₁²−k₂²+k₃²⁺k₄²)φk=0. This version is the so-called (2T) representation and given the signature (−,−,+,+).

When the permittivities, ε, are all equal, the wave equation is:

$$- \frac{{\partial }^{2} \vec{E}}{{c}^{2} \partial {t}^{2}} = \frac{1}{\overleftrightarrow{ɛ}} \vec{∇} \times \vec{∇} \times E \qquad ( 6 )$$

If the scalar extraordinary wave function is defined as φ=E, then the electromagnetic wave equation takes the following form:

$$\frac{{\partial }^{2} ϕ}{{c}^{2} \partial {t}^{2}} + \frac{{\partial }^{2} ϕ}{{ɛ}_{1} \partial {z}^{2}} + \frac{1}{{ɛ}_{2}} ( \frac{{\partial }^{2} ϕ}{\partial {x}^{2}} + \frac{{\partial }^{2} ϕ}{\partial {y}^{2}} ) \qquad ( 7 )$$

The analogies with phononic crystal elastic wave equation should now be clear to those skilled in the art.

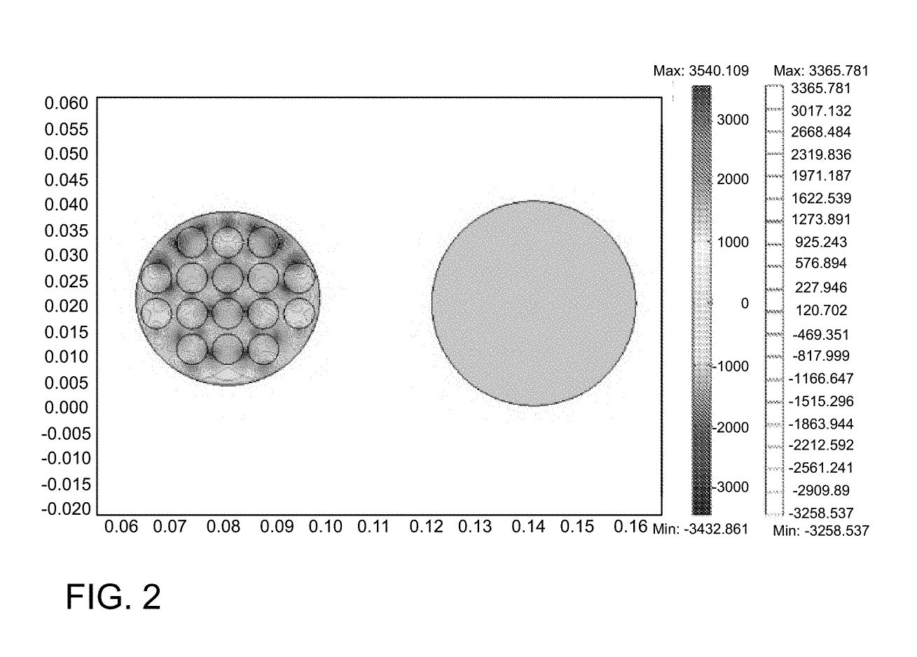

Turning now to FIG. 2, a screen dump from a finite element simulation of steel balls in glycerin surrounded by a circular acoustic transducer is shown. The circular structure on the left shows a ring transducer packed with steel balls and glycerin. In comparison, the circular structure on the right shows the same ring transducer filled with glycerin, but without any steel balls packed therein. The acoustic pressure is on an arbitrary scale. As seen on the scale, the difference in magnitude for the acoustic pressure between the two simulations is about three orders of magnitude. The distortions of the optical space created by high-pressure modulations in the fluid, specifically at the eigen-nodes in the phononic crystal as shown on the left-hand side of FIG. 2, create an effective hyperbolic space described identically with a Weierstrass function or by conventional optical dispersion relations.

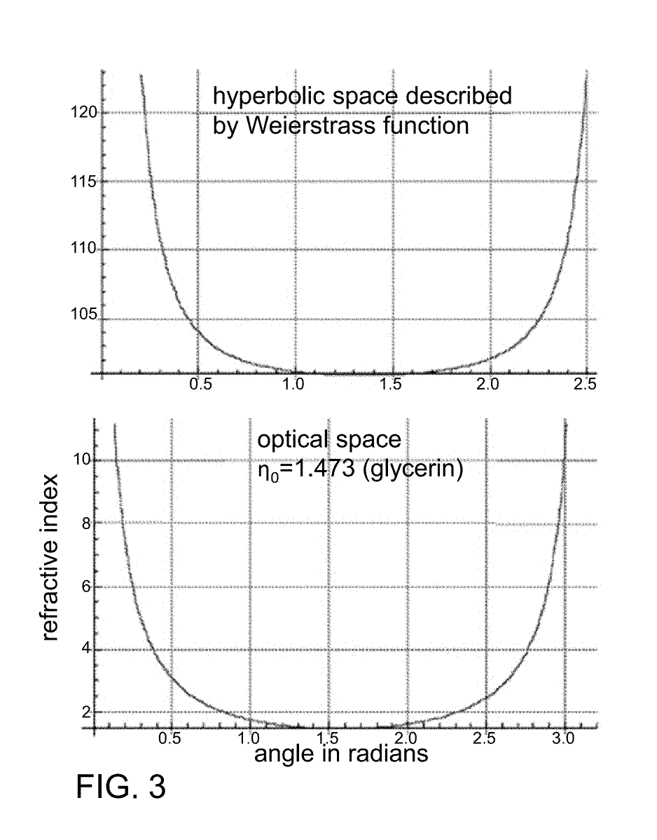

FIG. 3 shows simulations showing the hyperbolic surface similarity between optical space (bottom) and Minkowski space-time (top), with the optical space computed for glycerin as the acoustic transfer medium. The scale on the hyperbolic space is arbitrary, whereas the scale on the diagram of optical space is refractive index versus incident angle from zero to π radians with η₀ set to 1.472, the room temperature refractive index for glycerin.

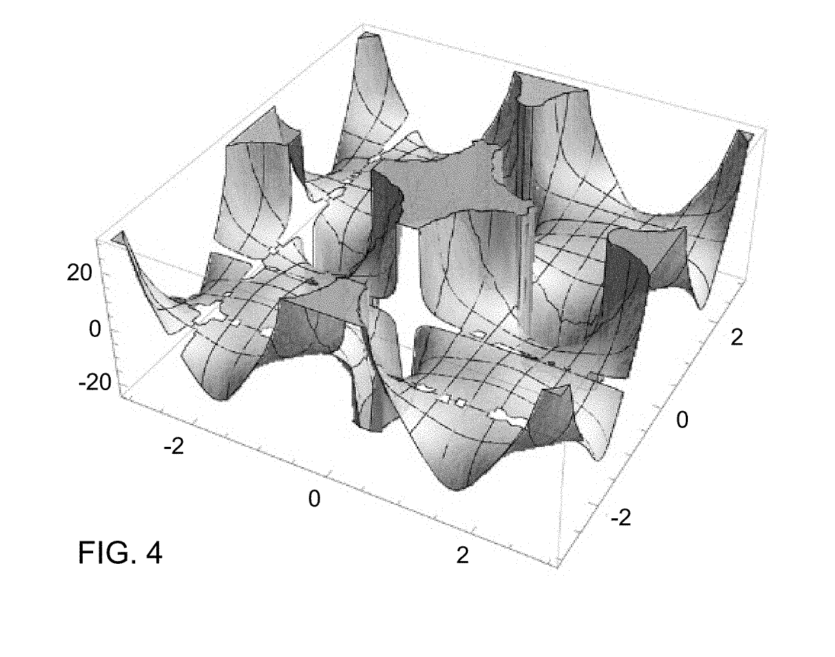

FIG. 4 shows a simulation of a three-dimensional hyperbolic surface showing warping of the space-time by large masses. In particular, FIG. 4 is a model of a hyperbolic surface computed with the Weierstrass function. In one-dimension this function is:

$$f ( x ) = {\sum }_{k = 1}^{∞} \frac{sin ( π {k}^{a} x )}{π {k}^{a}} \qquad ( 8 )$$

As seen in FIG. 4, this function is not exactly well-behaved, and the gaps on the surface are due to the fact that the function is not smoothly differentiable. It is differentiable on a set of points of measure zero. Nonetheless, the overall persistent homology on the manifold is clearly seen. There are deep wells in the surface very similar to the pressure nodes in the phononic crystal. These wells are not created by the same phenomena as the pressure nodes in the phononic crystal, but point to the actual acoustic-fluid dynamics taking place in the phononic crystal.

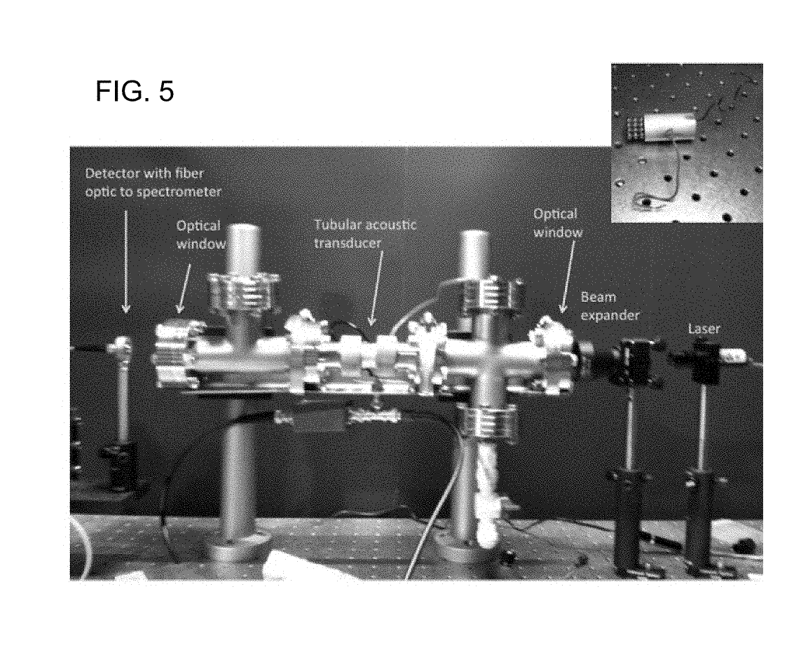

FIG. 5 illustrates a first exemplary system of the present disclosure. The system can be used to produce analog black holes and to measure the redshift. The system includes a red diode laser (˜365 nm) used to probe the interior of the phononic crystal. The light from the laser first enters a 5× beam spreader. The acoustic tube is a PZT-8 piezoelectric ceramic crystal with a resonance of 810 kHz, and connected to quick-connect fittings from Kurt J. Lesker®. The acoustic tube includes an inlet for filling the interior of the tube with acoustic fluid and the phononic crystal, and a separate port for internal electrical connections. The system also includes optical windows (made of quartz) on either end thereof. The light exiting the system is collected by a Thorlabs® F230SMA-B collimator and directed, via a fiber optic, into a Thorlabs® CCS spectrometer. The piezoceramic tube has an outer diameter of 2.252 cm, an inner diameter of 2.052 cm, and a length of 4.050 cm. The tube acts as the flow chamber in this device. The inset photo at the top right of FIG. 5 shows the phononic crystal, which is formed by a cylindrical frame that holds 0.5 mm magnetic steel balls arranged in a hexagonal array, prior to being inserted into the piezoacoustic tube.

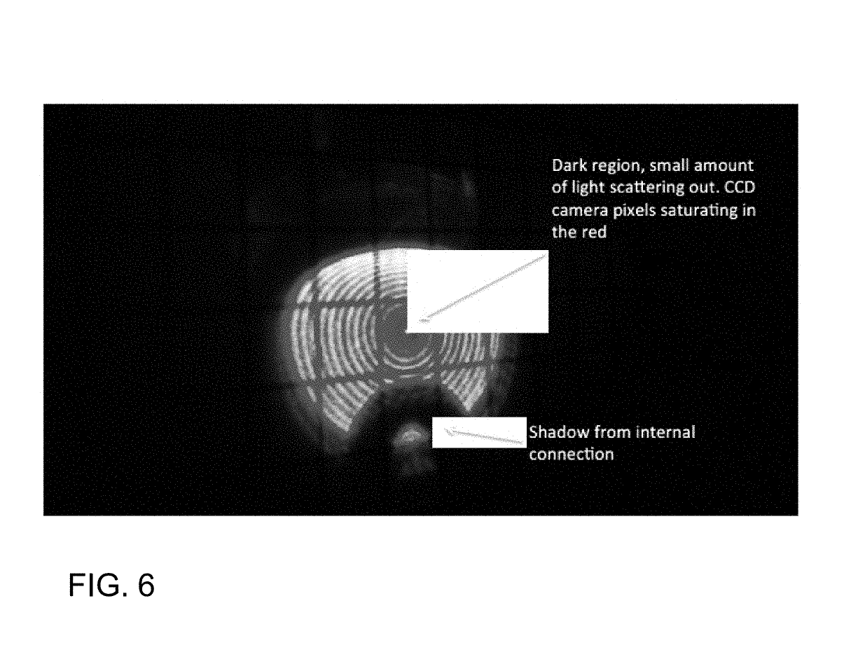

FIG. 6 shows light from the device of FIG. 5. Here, the device is powered at 1.62 MHz (second harmonic) and 10 Vpp without the phononic crystal inserted. The laser light is projected onto a centimeter grid, and the camera is at a slight viewing angle. Camera pixels are saturating, and there is a shadow at the bottom of the image of an internal connection to the piezotube. FIG. 6 shows the expected Bessel function behavior when powered (i.e., Bessel function optical rings are shown, similar to FIG. 1). Of course, the acoustic pressure is directly related to the voltage of the applied signal to the transducer.

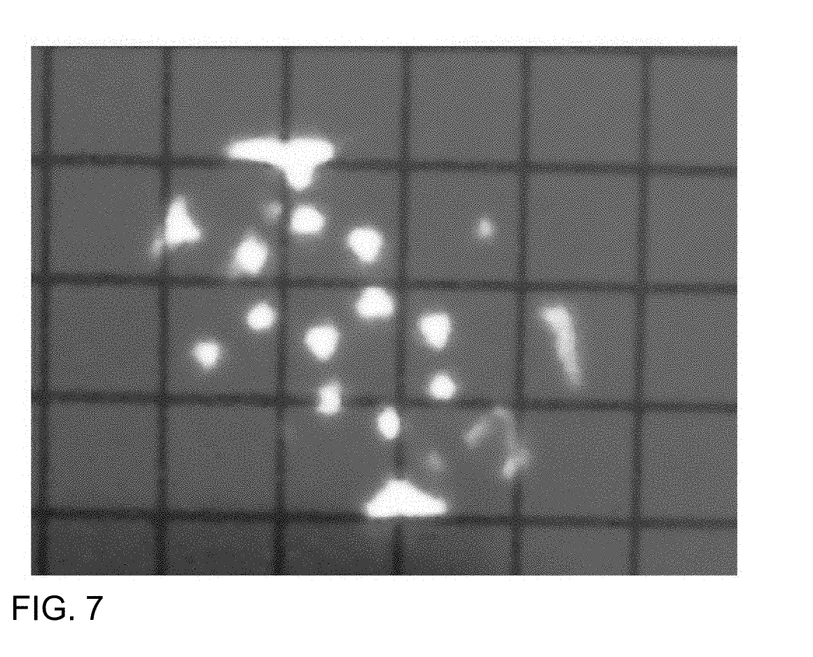

When the cavity within the tubular transducer (i.e. an active volume) is filled with a hexagonal-packed phononic crystal of 5 mm steel balls, such as in the inset image at the top right of FIG. 5, the image shown in FIG. 7 is produced. FIG. 7 particularly shows light shining out of the phononic crystal. The “blobs” seen in FIG. 7 are captured in the collimator and analyzed by the spectrometer to measure redshift. All of the main “blobs” in the hexagonal patterns had similar redshift, which is likely due to the high degree of symmetry in the experimental system. In this way, FIG. 7 shows the expected hexagonal pattern from the light passing through the interior of the crystal. The entire acoustic cavity and surrounding support plumbing is filled with glycerin to act as a dense acoustic transfer medium.

With phononic crystals, the optimal frequency (acoustic wavelength) to observe constructive and destructive interference occurs when the wavelength is about the same size as “atoms” in the crystal. In this case, the “atoms” are steel balls 5 mm in diameter. The frequency would then be 258 kHz. The piezotube has a primary resonance of 810 kHz. This arrangement suggests a subharmonic frequency of 202 kHz would be near optimal.

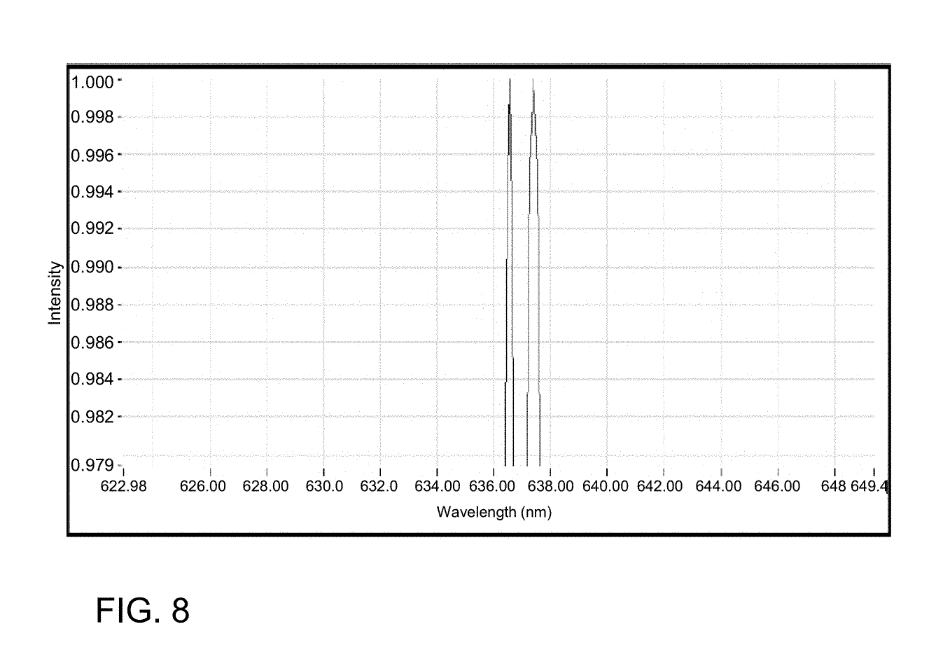

Experiments were done at a lower subharmonic frequency of 101 kHz (i.e., about half the optimal frequency) and at 10 Vpp square wave. When the transducer is powered, the expected constructive and destructive interference are obtained. To measure the change in pressure as a result of these constructive interferences, the light is captured in the spectrometer. FIG. 8 shows typical spectrometer data from one of the light “blobs” of FIG. 7. The blue line (left-hand side) in FIG. 8 is reference (i.e., the light prior to applying the acoustic drive frequency). As shown in FIG. 8, after applying the signal, the light is shifted roughly 0.8 nm (i.e., there is a redshift of 0.8 nm (+/−0.2)) to the right. The uncertainty is based on laser stability and averaged from several individual positions through the phononic crystal. The pressures are much higher, high enough to produce the redshift, not unlike that produced when light passes through a gravity well next to a planet-sized object. The difference between these two frequency lines divided by the reference is known as the redshift and is given by:

$$z = \frac{{λ}_{o} - {λ}_{e}}{{λ}_{e}} = 0.0013$$

The Schwarzschild radius near a black hole is given by:

$${R}_{s} = \frac{2 GM}{{c}^{2}}$$

where G is the gravitational constant; M is the mass of the cosmological object (e.g., black hole); and c is the speed of light. The redshift, z, is related through the following equation:

$$z = \frac{1}{\sqrt{1 - \frac{{R}_{s}}{r}}} - 1$$

where r is the radius of the hole, or, as will be shown herein, the radius of a planet.

Substituting and solving, the following equation is obtained:

$$\frac{M}{r} = \frac{1 - {( \frac{1}{1 + z} )}^{2}}{\frac{2 G}{{c}^{2}}}$$

Simplifying and substituting the constant values, the following equation is obtained:

$$\frac{M}{r} = z \frac{{c}^{2}}{G} = 0.013 \frac{8.94 \times {10}^{16}}{6.67 \times {10}^{- 11}} = 1.74 \times {10}^{24}$$

This equation gives the mass to radius in SI units. Thus, the “cosmological object” simulated in the lab has a mass to radius ratio similar to a planet. This status is deduced from the redshift.

It is now desirable to compute the pressure at these optical singularities. Using concepts from astrophysics and condensed matter physics, the pressure from the redshift can be calculated. When light travels from a distant galaxy, for example, it is redshifted. This shift in light perception is believed to be due to the expansion of the universe. A more modern explanation, at the cutting edge of cosmology, is that the light is slightly absorbed by interstellar matter and this absorption circumvents the dark energy explanation as the driving force for the expansion of the universe. Other possible causes for the observed cosmological redshift are given by the relationship

$$( 1 + {z}_{n} ) = ( \frac{n ( {t}_{o} )}{n ( {t}_{e} )} ) ( 1 + z )$$

where the z's are change in wavelengths and the n's are refractive index.

A more elementary equation comes from optics and is simply:

$${n}_{2} = {n}_{1} ( \frac{{λ}_{2}}{{λ}_{1}} ) = 1.4749 ⋆ ( \frac{637.4}{636.6} ) = 1.4768$$

which can be derived from the relation c=nᵢvᵢ.

In this equation, the numerical values of the wavelengths associated with the redshift and the refractive index have been inserted. Given c, two similar relations for different refractive indices and wavelengths can be equated. Using the wavelengths in FIG. 8 and the initial refractive index of glycerin as 1.4749, the new refractive index was computed to be 1.4768. Interpolating on an exponential fit of the data, the pressure is found to be 70 MPa at 10 Vpp and 101 kHz. As such, optical space singularities with properties similar to black holes have been demonstrated by inducing high pressure nodes in the interior of a phononic crystal with high-frequency sound.

Generally speaking, the device illustrated in FIG. 5 can be described as including a flow chamber with at least one inlet, and at least one ultrasonic transducer that surrounds an active volume within the flow chamber. In the device of FIG. 5, the flow chamber is defined by the tubular ultrasonic transducer. The active volume is the volume within the tubular ultrasonic transducer. A phononic crystal is located within the active volume.

The ultrasonic transducer does not have to be a tubular transducer. In some implementations, the ultrasonic transducer partially or completely surrounds the active volume. This arrangement could be done, for example, by using multiple transducers to surround the active volume, or by using a combination of transducers and reflectors to surround the active volume.

As seen in FIG. 5, the phononic crystal is in the form of a frame holding steel balls in a linear hexagonal array. Generally, the phononic crystal uses a periodic array, which can be other than hexagonal, for example cubic as in FIG. 2. The shape of the periodic array is viewed along a longitudinal axis. The objects used to form the periodic array do not have to be steel, and can generally be made of any metal, glass or ceramic. 3D-printed plastics and composites such as honeycomb constructed polyaramides could also be used for the objects in the periodic array. Generally, two materials with very different specific acoustic impedances should be used. One of the materials is the fluid, and the other material is provided by the phononic crystal. In particular embodiments, the periodic array is formed from a material having a specific acoustic impedance of greater than 15 E5 g/cm²·sec, or 15×10⁵ g/cm²·sec, which includes most metals. The frame is made of a material (e.g. a plastic) that has a specific acoustic impedance less than 4×10⁵ g/cm²·sec (4 E5 g/cm²·sec) (i.e. similar to the fluid).

The device includes two optical windows, one at either end of the device. A laser or other light source is located at one end of the device, and a collimator/light detector is located at the other end of the device. The laser illuminates the active volume of the flow chamber. A beam spreader may be used if desired.

Another embodiment of the acoustophoretic device is more suitable for continuous flow applications. The acoustophoretic device includes a flow chamber having at least one inlet and at least one outlet. The flow chamber includes an inlet, an outlet, and an ultrasonic transducer (tubular) surrounding the flow chamber. This device is oriented vertically, such that a fluid mixture flows vertically upwards from the at least one inlet toward the at least one outlet. Located within the flow chamber is an active volume, which is generally defined by the height of the ultrasonic transducer(s). A phononic crystal is located within the active volume.

The flow chamber is the region of the device through which is flowed a fluid, such as an initial mixture of a host fluid and a secondary phase/particulate. The inletis located at a first end of the flow chamber. In particular embodiments, the first end is a bottom end of the device. The outlet is located at a second end of the flow chamber, which can be a top end of the device. This outlet is usually a permeate outlet, i.e. used to recover the host fluid and residual secondary phase/particulate from the flow chamber. The ultrasonic transducer is between the inlet and the outlet.

The phononic crystal includes a frame that holds a periodic array of objects, such as balls. In this embodiment, the frame includes multiple layers. Each layer holds objects in a periodic structure, such as a hexagonal array. Thus, the phononic crystal here is balls arranged in a linear hexagonal array. The phononic crystal can of course have more layers, and a different periodic structure in each layer. The objects generally have a regular shape, such as spheres or other polygons. The distance between the objects is generally the same within each layer, and between layers as well. It is noted that fluid flowing through the flow chamber also flows through the phononic crystal.

In this embodiment, preferably, fluid flows through the device upwards. A mixture of host fluid containing secondary phase/particulate enters the device through the inlet at a bottom end of the device. The fluid mixture flows upwards through the flow chamber. There, the fluid mixture encounters the acoustic standing waves, which are used to separate the secondary phase/particulate from the host fluid. In particular, the devices disclosed herein generate very high-pressure nodes, which aids in the separation. Agglomeration, aggregation, clumping, or coalescence of the secondary phase/particulate occurs within the acoustic standing waves, which also concentrates the secondary phase/particulate. Host fluid, containing residual secondary phase/particulate not separated out, then exits through the permeate/flow outlet. As the secondary phase/particulate is concentrated, the concentrated clusters eventually overcome the combined effect of the fluid flow drag forces and acoustic radiation force, and they fall downwards, where they can be collected.

Many different liquids are contemplated as being used with the acoustophoretic devices of the present disclosure, all of which may serve as acoustic transfer mediums. For example, the fluid being flowed through the device may be a host fluid such as a cell culture medium, with the secondary phase/particulate including cells and other biomolecules. This fluid could be a cell-protein matrix suspension, with the proteins being the desirable material to separate and collect. Alternatively, the device of FIG. 5 might use glycerin or some other liquid, such as water, ketones, acetates, gels, or colloids as the acoustic transfer medium. Oil-and-water mixtures could be used, with the oil usually being suspended as the secondary phase in droplets of micron and sub-micron size.

Typically, the system is operated at a voltage/frequency such that the secondary phase/particles are trapped in the ultrasonic standing wave, i.e., remain in a stationary position. The axial component of the acoustic radiation force drives the particles, with a positive contrast factor, to the pressure nodal planes, whereas particles with a negative contrast factor are driven to the pressure anti-nodal planes. The radial or lateral component of the acoustic radiation force contributes to trapping the particle. The forces acting on a particle may be greater than the combined effect of fluid drag force and gravitational force. For small particles or emulsions, the drag force F_{D} can be expressed as:

$${\vec{F}}_{D} = 4 {πμ}_{f} {R}_{P} ( {\vec{U}}_{f} - {\vec{U}}_{p} ) [ \frac{1 + \frac{3}{2} \hat{μ}}{1 + \hat{μ}} ]$$

where U_{f} and Uₚ are the fluid and particle velocity, Rₚ is the particle radius, μ_{f} and μₚ are the dynamic viscosity of the fluid and particle, and \hat{μ}=μₚ/μ_{f} is the ratio of dynamic viscosities. The buoyancy force F_{B} is expressed as:

F_{B}=4/3πRₚ³(ρ_{f}−ρₚ)g

where Rₚ is the particle radius, ρ_{f} is the fluid density, ρₚ is the particle density, and g is the universal gravitational constant.

For a particle to be trapped in the ultrasonic standing wave, the force balance on the particle can be assumed to be zero, and therefore an expression for lateral acoustic radiation force F_{LRF} can be found, which is given by:

F_{LRF}=F_{D}+F_{B}

For a particle of known size and material property, and for a given flow rate, this equation can be used to estimate the magnitude of the lateral acoustic radiation force.

Particles with a positive contrast factor will be driven to the pressure nodal planes, and particles with a negative contrast factor will be driven to the pressure anti-nodal planes. In this way, the generation of a multi-dimensional acoustic standing wave in a flow chamber results in the creation of tightly packed clusters of particles in the flow chamber, typically corresponding to the location of the pressure nodes or anti-nodes in the standing wave depending on acoustic contrast factor.

The ultrasonic transducer(s) of the present disclosure produce acoustic standing waves, which can be planar acoustic standing waves or multi-dimensional acoustic standing waves. The multi-dimensional standing wave generates acoustic radiation forces in both the axial direction (i.e., in the direction of the standing wave, between the transducer and the reflector, perpendicular to the flow direction) and the lateral direction (i.e., in the flow direction). As the mixture flows through the flow chamber, particles in suspension experience a strong axial force component in the direction of the standing wave. Since this acoustic force is perpendicular to the flow direction and the drag force, it quickly moves the particles to pressure nodal planes or anti-nodal planes, depending on the contrast factor of the particle. The lateral acoustic radiation force then acts to move the concentrated particles towards the center of each planar node, resulting in agglomeration or clumping. The lateral acoustic radiation force component can overcome fluid drag for such clumps of particles to continually grow and then drop out of the mixture due to gravity. Therefore, both the drop in drag per particle as the particle cluster increases in size, as well as the drop in acoustic radiation force per particle as the particle cluster grows in size, may be considered in determining the effectiveness of the acoustic separator device. In the present disclosure, the lateral force component and the axial force component of the multi-dimensional acoustic standing wave are of the same order of magnitude. In this regard, it is noted that in a multi-dimensional acoustic standing wave, the axial force is stronger than the lateral force, but the lateral force of a multi-dimensional acoustic standing wave is much higher than the lateral force of a planar standing wave, usually by two orders of magnitude or more.

It may be helpful to provide an explanation now of how multi-dimensional acoustic standing waves can be generated. The multi-dimensional acoustic standing wave implemented for particle collection is obtained by driving an ultrasonic transducer at a frequency that both generates the acoustic standing wave and excites at least a fundamental 3D vibration mode of the piezoelectric material. Perturbation of the piezoelectric material in an ultrasonic transducer in a multimode fashion allows for generation of a multidimensional acoustic standing wave. A piezoelectric material, such as a crystal or poly-crystal, can be specifically designed to deform in a multimode fashion at designed frequencies, allowing for generation of a multi-dimensional acoustic standing wave. The multi-dimensional acoustic standing wave may be generated by distinct modes of the piezoelectric material such as a 3×3 mode that would generate multidimensional acoustic standing waves. A multitude of multidimensional acoustic standing waves may also be generated by allowing the piezoelectric material to vibrate through many different mode shapes. Thus, the material would excite multiple modes such as a 0×0 mode (i.e. a piston mode) to a 1×1, 2×2, 1×3, 3×1, 3×3, and other higher order modes and then cycle back through the lower modes of the material (not necessarily in straight order). This switching or dithering of the material between modes allows for various multidimensional wave shapes, along with a single piston mode shape to be generated over a designated time.

Some further explanation of the ultrasonic transducers used in the devices, systems, and methods of the present disclosure may be helpful as well. In this regard, the transducers use a piezoelectric material, such as a crystal or poly-crystal, usually made of PZT-8 (lead zirconate titanate). Such crystals may have a 1 inch diameter and a nominal 2 MHz resonance frequency, and may also be of a larger size. Each ultrasonic transducer module can have only one crystal, or can have multiple crystals that each act as a separate ultrasonic transducer and are either controlled by one or multiple amplifiers. The piezoelectric material can be square, rectangular, irregular polygon, or generally of any arbitrary shape. The transducer(s) is/are used to create a pressure field that generates forces of the same order of magnitude both orthogonal to the standing wave direction (lateral) and in the standing wave direction (axial).

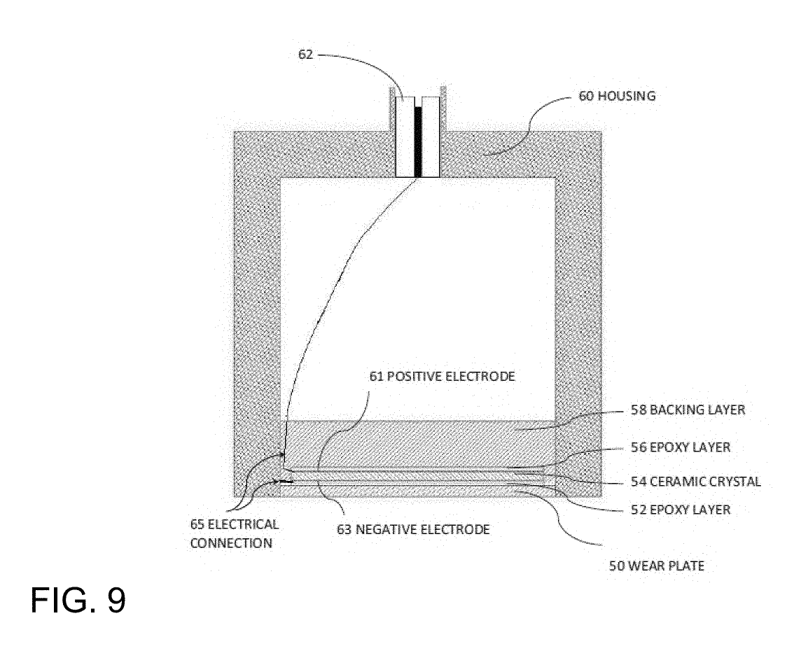

FIG. 9 is a cross-sectional diagram of a conventional ultrasonic transducer. This transducer has a wear plate 50 at a bottom end, epoxy layer 52, ceramic crystal 54 (made of, e.g. PZT), an epoxy layer 56, and a backing layer 58. On either side of the ceramic crystal, there is an electrode: a positive electrode 61 and a negative electrode 63. The epoxy layer 56 attaches backing layer 58 to the crystal 54. The entire assembly is contained in a housing 60 which may be made out of, for example, aluminum. An electrical adapter 62 provides connection for wires to pass through the housing and connect to leads (not shown) which attach to the crystal 54. Typically, backing layers are designed to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

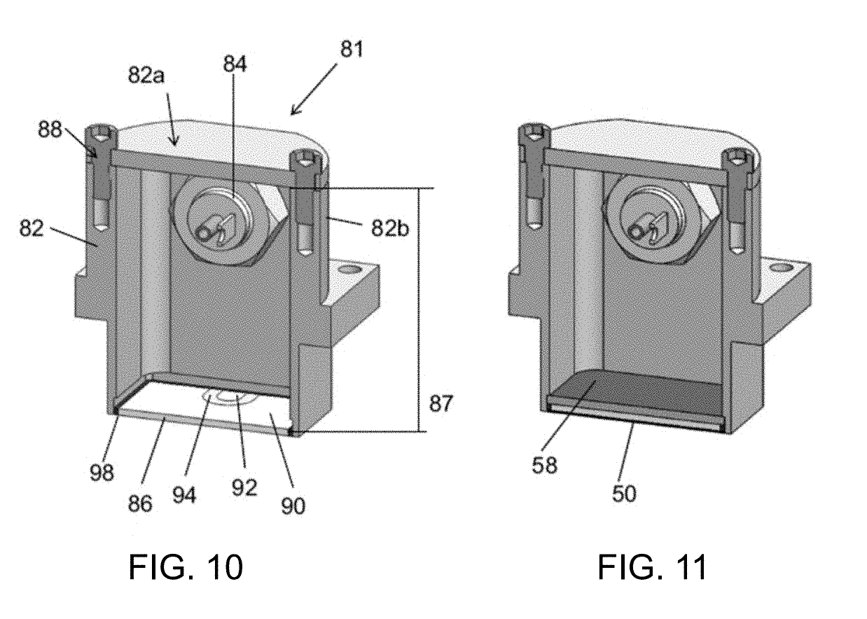

FIG. 10 is a cross-sectional view of an ultrasonic transducer 81 of the present disclosure. Transducer 81 is shaped as a disc or a plate, and has an aluminum housing 82. The piezoelectric material is a mass of perovskite ceramic crystals, each consisting of a small, tetravalent metal ion, usually titanium or zirconium, in a lattice of larger, divalent metal ions, usually lead or barium, and O2-ions. As an example, a PZT (lead zirconate titanate) crystal 86 defines the bottom end of the transducer, and is exposed from the exterior of the housing. The crystal has an interior surface and an exterior surface. The crystal is supported on its perimeter by a small elastic layer 98, e.g. silicone or similar material, located between the crystal and the housing. Put another way, no wear layer is present. In particular embodiments, the crystal is an irregular polygon, and in further embodiments is an asymmetrical irregular polygon.

Screws 88 attach an aluminum top plate 82a of the housing to the body 82b of the housing via threads. The top plate includes a connector 84 for powering the transducer. The top surface of the PZT crystal 86 is connected to a positive electrode 90 and a negative electrode 92, which are separated by an insulating material 94. The electrodes can be made from any conductive material, such as silver or nickel. Electrical power is provided to the PZT crystal 86 through the electrodes on the crystal. Note that the crystal 86 has no backing layer or epoxy layer. Put another way, there is an air gap 87 in the transducer between aluminum top plate 82a and the crystal 86 (i.e. the air gap is completely empty). A minimal backing 58 (on the interior surface) and/or wear plate 50 (on the exterior surface), as seen in FIG. 11.

The transducer design can affect performance of the system. A typical transducer is a layered structure with the ceramic crystal bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the piezoelectric material to vibrate in one of its eigenmodes (i.e. near eigenfrequency) with a high Q-factor. The vibrating ceramic crystal/disk is directly exposed to the fluid flowing through the flow chamber.

Removing the backing (e.g. making the piezoelectric material air backed) also permits the ceramic crystal to vibrate at higher order modes of vibration with little damping (e.g. higher order modal displacement). In a transducer having a piezoelectric material with a backing, the piezoelectric material vibrates with a more uniform displacement, like a piston. Removing the backing allows the piezoelectric material to vibrate in a non-uniform displacement mode. The higher order the mode shape of the piezoelectric material, the more nodal lines the piezoelectric material has. The higher order modal displacement of the piezoelectric material creates more trapping lines, although the correlation of trapping line to node is not necessarily one to one, and driving the piezoelectric material at a higher frequency will not necessarily produce more trapping lines.

In some embodiments, the piezoelectric material may have a backing that minimally affects the Q-factor of the piezoelectric material (e.g. less than 5%). The backing may be made of a substantially acoustically transparent material such as balsa wood, foam, or cork which allows the piezoelectric material to vibrate in a higher order mode shape and maintains a high Q-factor while still providing some mechanical support for the piezoelectric material. The backing layer may be a solid, or may be a lattice having holes through the layer, such that the lattice follows the nodes of the vibrating piezoelectric material in a particular higher order vibration mode, providing support at node locations while allowing the rest of the piezoelectric material to vibrate freely. The goal of the lattice work or acoustically transparent material is to provide support without lowering the Q-factor of the piezoelectric material or interfering with the excitation of a particular mode shape.

Placing the piezoelectric material in direct contact with the fluid also contributes to the high Q-factor by avoiding the dampening and energy absorption effects of the epoxy layer and the wear plate. Other embodiments may have wear plates or a wear surface to prevent the PZT, which contains lead, contacting the host fluid. This additional layer may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymers or polymer films. Organic and biocompatible coatings such as silicone or polyurethane are also usable as a wear surface.

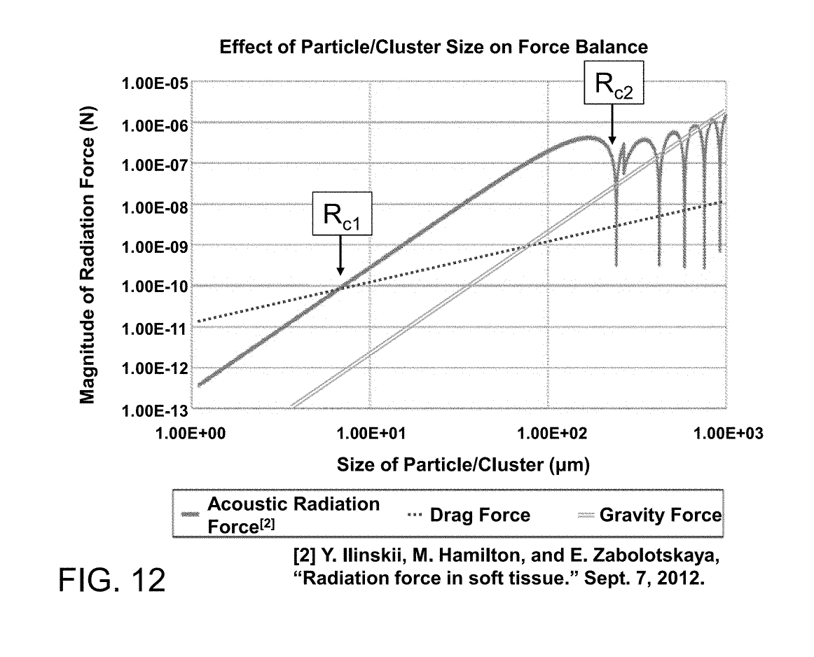

FIG. 12 is a log-log graph (logarithmic y-axis, logarithmic x-axis) that shows the scaling of the acoustic radiation force, fluid drag force, and buoyancy force with particle radius, and provides an explanation for the separation of particles using acoustic radiation forces. The buoyancy force is a particle volume dependent force, and is therefore negligible for particle sizes on the order of micron, but grows, and becomes significant for particle sizes on the order of hundreds of microns. The fluid drag force (Stokes drag force) scales linearly with fluid velocity, and therefore typically exceeds the buoyancy force for micron sized particles, but is negligible for larger sized particles on the order of hundreds of microns. The acoustic radiation force scaling is different. When the particle size is small, Gor'kov's equation is accurate and the acoustic trapping force scales with the volume of the particle. Eventually, when the particle size grows, the acoustic radiation force no longer increases with the cube of the particle radius, and will rapidly vanish at a certain critical particle size. For further increases of particle size, the radiation force increases again in magnitude but with opposite phase (not shown in the graph). This pattern repeats for increasing particle sizes.

Initially, when a suspension is flowing through the system with primarily small micron sized particles, the acoustic radiation force can balance the combined effect of fluid drag force and buoyancy force to permit a particle to be trapped in the standing wave. In FIG. 12, this balance happens at a particle size labeled as R_{c1}. The graph then indicates that all larger particles will be trapped as well. Therefore, when small particles are trapped in the standing wave, particles coalescence/clumping/aggregation/agglomeration takes place, resulting in continuous growth of effective particle size. As particles cluster, the total drag on the cluster is much lower than the sum of the drag forces on the individual particles. In essence, as the particles cluster, they shield each other from the fluid flow and reduce the overall drag of the cluster. As the particle cluster size grows, the acoustic radiation force reflects off the cluster, such that the net acoustic radiation force decreases per unit volume. The acoustic lateral forces on the particles may be greater than the drag forces to permit the clusters to remain stationary and grow in size.

Particle size growth continues until the buoyancy force becomes dominant, which is indicated by a second critical particle size, R_{c2}. The buoyancy force per unit volume of the cluster remains constant with cluster size, since it is a function of the particle density, cluster concentration and gravity constant. Therefore, as the cluster size increases, the buoyancy force on the cluster increases faster than the acoustic radiation force. At the size R_{c2}, the particles will rise or sink, depending on their relative density with respect to the host fluid. At this size, acoustic forces are secondary, gravity/buoyancy forces become dominant, and the particles naturally drop out or rise out of the host fluid. Not all particles will drop out, and those remaining particles and new particles entering the flow chamber will continue to move to the three-dimensional nodal locations, repeating the growth and drop-out process. This phenomenon explains the quick drops and rises in the acoustic radiation force beyond size R_{c2}. Thus, FIG. 12 explains how small particles can be trapped continuously in a standing wave, grow into larger particles or clumps, and then eventually will rise or settle out because of increased buoyancy force.

The size, shape, and thickness of the transducer determine the transducer displacement at different frequencies of excitation, which in turn affects particle separation efficiency. Higher order modal displacements generate three-dimensional acoustic standing waves with strong gradients in the acoustic field in all directions, thereby creating equally strong acoustic radiation forces in all directions, leading to multiple trapping lines, where the number of trapping lines correlate with the particular mode shape of the transducer.

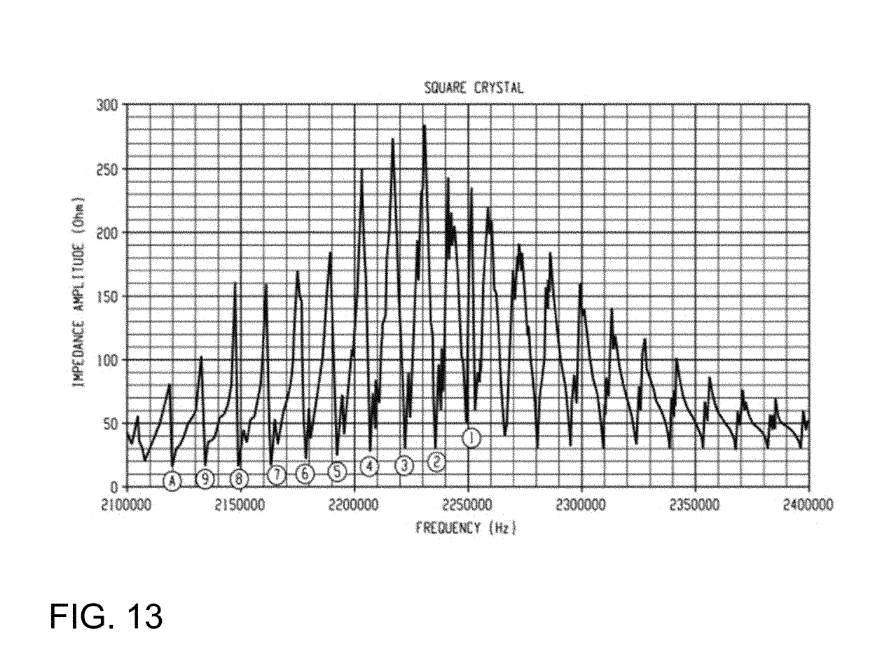

FIG. 13 shows the measured electrical impedance amplitude of the transducer as a function of frequency in the vicinity of the 2.2 MHz transducer resonance. The minima in the transducer electrical impedance correspond to acoustic resonances of a water column and represent potential frequencies for operation. Numerical modeling has indicated that the transducer displacement profile varies significantly at these acoustic resonance frequencies, and thereby directly affects the acoustic standing wave and resulting trapping force. Since the transducer operates near its thickness resonance, the displacements of the electrode surfaces are essentially out of phase. The typical displacement of the transducer electrodes is not uniform and varies depending on frequency of excitation. Higher order transducer displacement patterns result in higher trapping forces and multiple stable trapping lines for the captured particles.

To investigate the effect of the transducer displacement profile on acoustic trapping force and particle separation efficiencies, an experiment was repeated ten times, with all conditions identical except for the excitation frequency. Ten consecutive acoustic resonance frequencies, indicated by circled numbers 1-9 and letter A on FIG. 13, were used as excitation frequencies. The conditions were experiment duration of 30 min, a 1000 ppm oil concentration of approximately 5-micron SAE-30 oil droplets, a flow rate of 500 ml/min, and an applied power of 20 W.

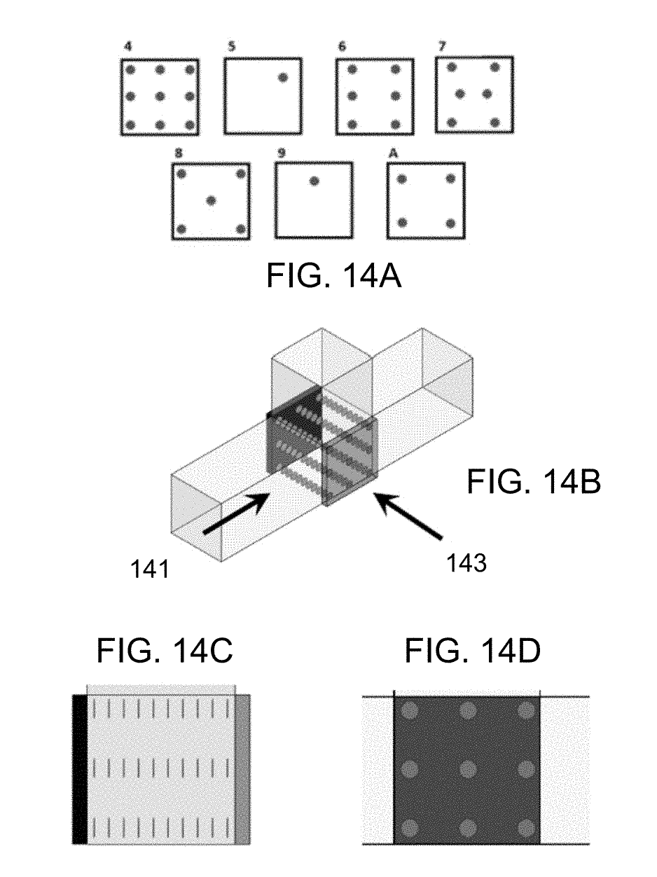

As the emulsion passed by the transducer, the trapping lines of oil droplets were observed and characterized. The characterization involved the observation and pattern of the number of trapping lines across the fluid channel, as shown in FIG. 14A, for seven of the ten resonance frequencies identified in FIG. 13.

FIG. 14B shows an isometric view of the device in which the trapping line locations are being determined. FIG. 14C is a view of the device as it appears when looking down the inlet, along arrow 141. FIG. 14D is a view of the device as it appears when looking directly at the transducer face, along arrow 143.

The effect of excitation frequency clearly determines the number of trapping lines, which vary from a single trapping line at the excitation frequency of acoustic resonance 5 and 9, to nine trapping lines for acoustic resonance frequency 4. At other excitation frequencies four or five trapping lines are observed. Different displacement profiles of the transducer can produce different (more) trapping lines in the standing waves, with more gradients in displacement profile generally creating higher trapping forces and more trapping lines. It is noted that although the different trapping line profiles shown in FIG. 14A were obtained at the frequencies shown in FIG. 13, these trapping line profiles can also be obtained at different frequencies.

FIG. 14A shows the different crystal vibration modes possible by driving the crystal to vibrate at different fundamental frequencies of vibration. The 3D mode of vibration of the crystal is carried by the acoustic standing wave across the fluid in the chamber all the way to the reflector and back. The resulting multi-dimensional standing wave can be thought of as containing two components. The first component is a planar out-of-plane motion component (uniform displacement across crystal surface) of the crystal that generates a standing wave, and the second component is a displacement amplitude variation with peaks and valleys occurring in both lateral directions of the crystal surface. Three-dimensional force gradients are generated by the standing wave. These three-dimensional force gradients result in lateral radiation forces that stop and trap the particles with respect to the flow by overcoming the viscous drag force. In addition, the lateral radiation forces are responsible for creating tightly packed clusters of particles. Therefore, particle separation and gravity-driven collection depends on generating a multi-dimensional standing wave that can overcome the particle drag force as the mixture flows through the acoustic standing wave. Multiple particle clusters are formed along trapping lines in the axial direction of the standing wave, as presented schematically in FIG. 14A.

The devices of the present disclosure can be used to provide physical setups that are similar to black holes, for use as a cosmological model. They can also be used for sonochemistry. Sonochemistry results when the acoustic standing waves generate high-pressure nodes within the active volume. Acoustic cavitation (the formation, growth, and implosion of bubbles within the fluid) can occur. The high-pressure nodes have a high amount of energy, which result in increased chemical reaction between reactants.

Another application is the destruction of materials in the fluid stream by moving such materials towards the high-pressure nodes within the device. For example, proteins can be denatured at the high-pressure nodes to deactivate biological molecules within the fluid stream. Another application is to separate materials in the fluid stream from the fluid itself, to obtain clarified/purified fluid.

It is contemplated that the acoustophoretic systems/devices of the present disclosure can be used in a filter “train,” in which multiple different filtration steps are used to clarify or purify an initial fluid/particle mixture to obtain the desired product and manage different materials from each filtration step. Each filtration step can be optimized to remove a particular material, improving the overall efficiency of the clarification process. An individual acoustophoretic device can operate as one or multiple filtration steps. For example, each individual ultrasonic transducer within a particular acoustophoretic device can to operated to trap materials within a given particle range. It is particularly contemplated that the acoustophoretic device can be used to remove large quantities of material, reducing the burden on subsequent downstream filtration steps/stages. However, it is contemplated that additional filtration steps/stages can be placed upstream or downstream of the acoustophoretic device. Of course, multiple acoustophoretic devices can be used as well. It is particularly contemplated that desirable biomolecules or cells can be recovered/separated after such filtration/purification.

The outlets of the acoustophoretic devices of the present disclosure (e.g. clarified fluid and concentrated cells) can be fluidly connected to any other filtration step or filtration stage. Such filtration steps can include various methods such as depth filtration, sterile filtration, size exclusion filtration, or tangential filtration. Depth filtration uses physical porous filtration mediums that can retain material through the entire depth of the filter. In sterile filtration, membrane filters with extremely small pore sizes are used to remove microorganisms and viruses, generally without heat or irradiation or exposure to chemicals. Size exclusion filtration separates materials by size and/or molecular weight using physical filters with pores of given size. In tangential filtration, the majority of fluid flow is across the surface of the filter, rather than into the filter.

Chromatography can also be used, including cationic chromatography columns, anionic chromatography columns, affinity chromatography columns, mixed bed chromatography columns. Other hydrophilic/hydrophobic processes can also be used for filtration purposes.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. An acoustophoretic device, comprising:
a flow chamber including at least one inlet;
at least one ultrasonic transducer around at least a portion of an active volume within the flow chamber, the at least one ultrasonic transducer including a piezoelectric material configured to be driven to create an acoustic standing wave in the active volume; and
a phononic crystal within the active volume, wherein the phononic crystal occupies less than the entirety of the active volume.

2. The acoustophoretic device of claim 1, wherein the phononic crystal is in the form of a frame that supports a periodic array of objects, the objects being formed from a material with a specific acoustic impedance of greater than 15×10⁵ g/cm²·sec.

3. The acoustophoretic device of claim 2, wherein the objects are made of steel, another metal, glass or ceramic.

4. The acoustophoretic device of claim 2, wherein the frame is made of a material with a specific acoustic impedance of less than 4×10⁵ g/cm²·sec.

5. The acoustophoretic device of claim 2, wherein the periodic array is in the form of a linear hexagonal array or a cubic array.

6. The acoustophoretic device of claim 1, wherein the at least one ultrasonic transducer is a tubular ultrasonic transducer.

7. The acoustophoretic device of claim 1, wherein the flow chamber further comprises at least one outlet.

8. The acoustophoretic device of claim 7, wherein the at least one inlet is located at a first end of the flow chamber, and the at least one outlet is located at a second end of the flow chamber opposite the first end.

9. The acoustophoretic device of claim 1, further comprising an acoustic transfer medium within the active volume.

10. The acoustophoretic device of claim 9, wherein the acoustic transfer medium is glycerin, water, or oil.

11. The acoustophoretic device of claim 1, further comprising a first optical window at a first end of the flow chamber, and a second optical window located at a second end of the flow chamber opposite the first end.

12. The acoustophoretic device of claim 11, further comprising a laser located so as to illuminate the active volume through the first optical window; and a collimator located so as to receive light through the second optical window.

13. The acoustophoretic device of claim 12, wherein the light detector is coupled to a spectrometer.

14. The acoustophoretic device of claim 12, further comprising a beam expander between the laser and the first optical window.

15. A method for separating a secondary phase or a particulate in a host fluid, the method comprising:
flowing through an acoustophoretic device a mixture of the host fluid and the secondary phase or particulate, the acoustophoretic device comprising:
a flow chamber including at least one inlet;
at least one ultrasonic transducer around at least a portion of an active volume within the flow chamber, the at least one ultrasonic transducer including a piezoelectric material configured to be driven to create an acoustic standing wave in the active volume; and
a phononic crystal within the active volume, wherein the phononic crystal occupies less than the entirety of the active volume;
wherein the mixture fills the remainder of the active volume; and
driving the at least one ultrasonic transducer to create the acoustic standing wave in the active volume, wherein the secondary phase or particulate is driven to high pressure nodes in the active volume based on their acoustic contrast factor, and wherein the pressure of the high pressure nodes is at least 50 MPa.

16. The method of claim 15, wherein the host fluid is water and the secondary phase is oil; or wherein the host fluid is a cell culture medium and the secondary phase is proteins.

17. A method for performing sonochemistry between at least two reactants in a host fluid, the method comprising:
flowing through an acoustophoretic device a mixture of the host fluid and the at least two reactants, the acoustophoretic device comprising:
a flow chamber including at least one inlet;
at least one ultrasonic transducer around at least a portion of an active volume within the flow chamber, each ultrasonic transducer including a piezoelectric material configured to be driven to create an acoustic standing wave in the active volume; and
a phononic crystal within the active volume, wherein the phononic crystal occupies less than the entirety of the active volume;
wherein the mixture fills the remainder of the active volume; and
driving the at least one ultrasonic transducer to create the acoustic standing wave in the active volume, resulting in the creation of high pressure nodes with a pressure of at least 50 MPa.

18. A method for creating high pressure nodes with a pressure of at least 50 MPa, the method comprising:
receiving an acoustophoretic device that comprises:
a flow chamber including at least one inlet;
at least one ultrasonic transducer around at least a portion of an active volume within the flow chamber, each ultrasonic transducer including a piezoelectric material configured to be driven to create an acoustic standing wave in the active volume; and
a phononic crystal within the active volume, wherein the phononic crystal occupies less than the entirety of the active volume;
filling the remainder of the active volume with an acoustic transfer fluid; and
driving the at least one ultrasonic transducer to create the acoustic standing wave in the active volume, resulting in the creation of the high pressure nodes with a pressure of at least 50 MPa.

19. The method of claim 18, wherein the phononic crystal is in the form of a frame that supports a periodic array of steel balls.

20. The method of claim 18, wherein the acoustic transfer fluid is glycerin.
